(19) United States
(12) Patent Application Publication
(10) Pub. No.: US 2026/0046381 A1
(43) Pub. Date: Feb. 12, 2026

(54) SYSTEM AND METHOD FOR VASCULAR EFFECTS

(71) Applicant: Universal City Studios LLC, Universal City, CA (US)

(72) Inventors: Gregory Rell, Orlando, FL (US); Gregory Hall, Orlando, FL (US); Stephen Ketchum, Orlando, FL (US)

(21) Appl. No.: 18/797,147

(22) Filed: Aug. 7, 2024

(51) Int. Cl. H04N 9/31; A61B 5/00; G06T 11/00; G06T 13/80

(52) U.S. Cl. CPC H04N 9/3179; A61B 5/489; G06T 11/00; G06T 13/80

(57) ABSTRACT

A method of providing vascular effects includes emitting infrared light toward a body part of a guest, receiving the infrared light from the body part of the guest, generating a signal indicative of the received infrared light, generating a set of coordinates corresponding to a vascular structure of the body part of the guest based on the signal indicative of the received infrared light, mapping the vascular structure to the body part of the guest, retrieving a portion of code representing a sprite, generating a series of images of the vascular structure of the body part of the guest and the sprite, and projecting the series of images of the vascular structure and the sprite onto the body part of the guest.

### BACKGROUND

The present disclosure relates generally to providing interactive experiences in curated environments.

Interactive experiences can encourage guest engagement and satisfaction in curated environments, such as amusement parks, museums, historical sites, zoos, parks, art galleries, fairs, trade shows, conferences, conventions, expos, festivals, and so forth. Accordingly, new techniques for providing interactive experiences in curated environments are needed in order to increase guest engagement.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION

Certain embodiments commensurate in scope with the originally claimed subject matter are summarized below. These embodiments are not intended to limit the scope of the disclosure, but rather these embodiments are intended only to provide a brief summary of certain disclosed embodiments. Indeed, the present disclosure may encompass a variety of forms that may be similar to or different from the embodiments set forth below

In an embodiment, a vascular effects system includes an emitter, a receiver, a projector, processing circuitry, and memory. The emitter emits infrared light toward a body part of a guest. The receiver receives the infrared light from the body part of the guest and generates a signal indicative of the received infrared light. The projector projects images onto the body part of the guest. The memory stores instructions that, when executed by the processing circuitry, cause the processing circuitry to receive the signal indicative of the received infrared light from the receiver, generate a set of coordinates corresponding to a vascular structure of the body part of the guest based on the signal indicative of the received infrared light, retrieve the portion of code representing the sprite from the memory, generate an image of the vascular structure and the sprite, and project the image of the vascular structure and the sprite onto the body part of the guest via the projector.

In an embodiment, a method of providing vascular effects includes emitting infrared light toward a body part of a guest, receiving the infrared light from the body part of the guest, generating a signal indicative of the received infrared light, generating a set of coordinates corresponding to a vascular structure of the body part of the guest based on the signal indicative of the received infrared light, mapping the vascular structure to an area of the body part of the guest, retrieving a portion of code representing a sprite, generating a series of images of the vascular structure of the area of the body part of the guest and the sprite, and projecting the series of images of the vascular structure and the sprite onto the body part of the guest.

In an embodiment, a non-transitory computer readable medium stores instructions that, when executed by processing circuitry, cause the processing circuitry to receive a signal indicative of infrared light sensed from a body part of a guest, generate a set of coordinates corresponding to a vascular structure of the body part of the guest based on the signal indicative of the sensed infrared light, map the vascular structure to an area of the body part of the guest, retrieve a portion of code representing a sprite, generate an image of the vascular structure of the area of the body part of the guest and the sprite, and transmit the image to a projector to project the image onto the body part of the guest.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic of a vascular effects system being used in an amusement park, in accordance with an embodiment of the present disclosure;

FIG. 2 is a schematic of the vascular effects system of FIG. 1, in accordance with an embodiment of the present disclosure;

FIG. 3A illustrates an example in which the vascular effects system of FIGS. 1 and 2 depicts one or more bugs crawling along veins of a vascular structure, in accordance with an embodiment of the present disclosure;

FIG. 3B illustrates an example in which the vascular effects system of FIGS. 1 and 2 depicts magic flowing through the veins of the vascular structure, in accordance with an embodiment of the present disclosure;

FIG. 3C illustrates an example in which the vascular effects system of FIGS. 1 and 2 depicts fluid flowing through the veins of the vascular structure, in accordance with an embodiment of the present disclosure;

FIG. 3D illustrates an example in which the vascular effects system of FIGS. 1 and 2 depicts the veins of the vascular structure being larger on one side of a line, in accordance with an embodiment of the present disclosure;

FIG. 4 is a block diagram of example components of a computing device that could be used in the vascular effects system of FIGS. 1 and 2, or some other device of FIGS. 1 and 2, in accordance with an embodiment of the present disclosure; and

FIG. 5 is a flowchart of a process for providing vascular effects, in accordance with an embodiment of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers’ specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Furthermore, the particular features, structures, or characteristics may be combined in any suitable manner in one or more embodiments.

The present disclosure is directed to techniques for creating vascular effects in a curated environment (e.g., amusement parks, museums, historical sites, zoos, parks, art galleries, fairs, trade shows, conferences, conventions, expos, festivals, and so forth) using a vascular effects system. The vascular effects system includes an emitter, a receiver, a projector, and a processor-based computing device. The emitter emits infrared light onto a body part of a guest, such as an arm, a hand, or a leg. The receiver detects the infrared light from the body part of the guest and outputs a signal that may be processed to identify the vascular structure of the body part of a guest underneath the skin. The computing device receives the signal from the receiver, generates two-dimensional coordinates of the vascular structure, and maps the vascular structure to an area to be projected onto the guest’s body part. The computing device retrieves code defining one or more sprites from memory and adds the sprites to the projection. The sprites may include images and/or animations that move along the vascular structure. For example, the sprites may represent bugs crawling on the guest’s veins, tattoos on the guest’s skin, magic, superpowers, or other supernatural forces/substances flowing through the guest’s veins, various colors of blood or other fluids flowing through the guest’s veins, poison flowing through the guest’s veins, veins getting bigger (e.g., swelling) or smaller, veins pulsing, veins moving around, and so forth. As the guest’s body part moves relative to the vascular effect system, the projected images may update to show a different portion of the guest’s vascular structure.

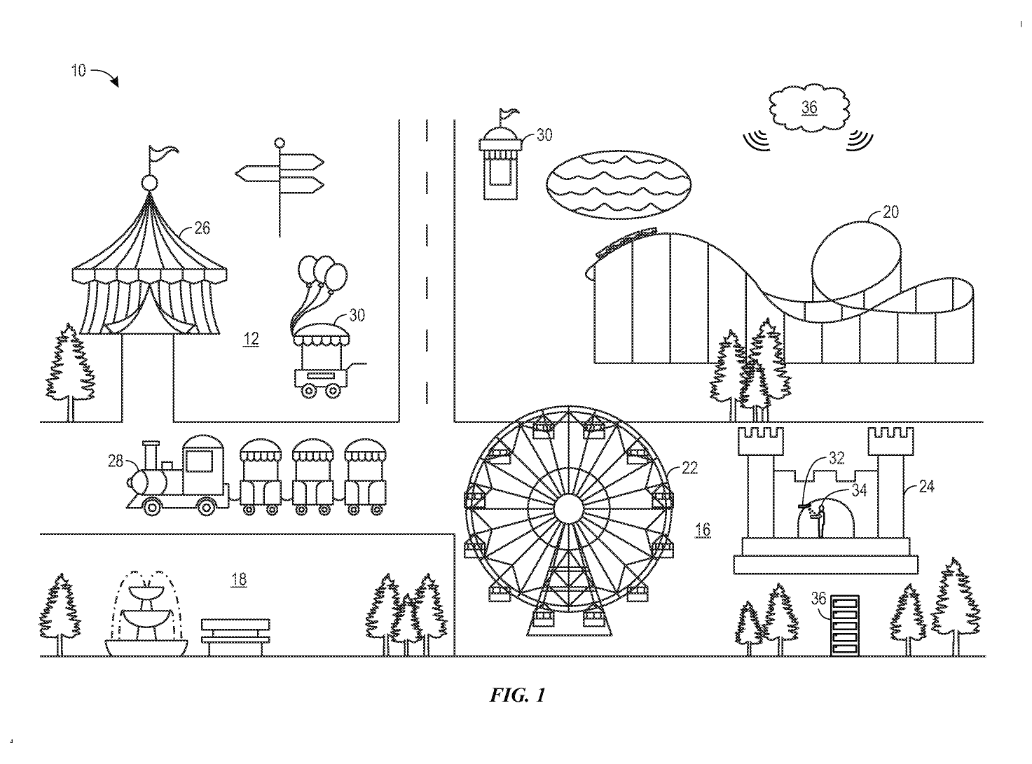

FIG. 1 is a schematic of an amusement park 10. The amusement park 10 may include and/or be separated into one or more sections or lands, such as a first land 12, a second land 14, a third land 16, and a fourth land 18. Each of the lands 12, 14, 16, 18 may include one or more attractions. As shown in FIG. 1, the attractions may include rides, such as roller coasters 20, carousels 22, or attractions in which a guest is moved through an environment, environments through which guests walk, such as castles 24, performance venues 26, and so forth. The amusement park 10 may also include transportation 28, such as trams, trains, trolleys, and so forth that are configured to move guests within or between lands 12, 14, 16, 18 of the amusement park 10. Further, the amusement park 10 may include one or more vending locations 30. The vending locations 30 may be stationary (e.g., a storefront), mobile (e.g., a cart), or semi-mobile (e.g., a stand), and configured to sell items, such as food, merchandise, toys, souvenirs, toiletries, and so forth to guests.

Some of the attractions at the amusement park 10 may include interactive components to improve the guest experience and increase guest engagement. For example, as shown in FIG. 1, the castle 24 may include an interactive vascular effects system 32. A guest 34 may put a body part under the vascular effects system 32 and the vascular effects system 32 may, utilizing one or more servers 36, project images on the guest’s body that recreate the vascular structure of the guest’s body part and may add one or more effects to the recreated vascular structure. For example, the vascular effects system 32 may simulate bugs crawling on the guest’s veins, tattoos on the guest’s skin, magic, superpowers, or other supernatural forces/substances flowing through the guest’s veins, various colors of blood or other fluids flowing through the guest’s veins, poison flowing through the guest’s veins, veins getting bigger (e.g., swelling, growing, sprouting smaller veins) or smaller, veins moving around, and so forth. In some embodiments, the vascular effects system 32 may also use haptics (e.g., small puffs of air onto the guest’s skin to enhance the vascular effects.

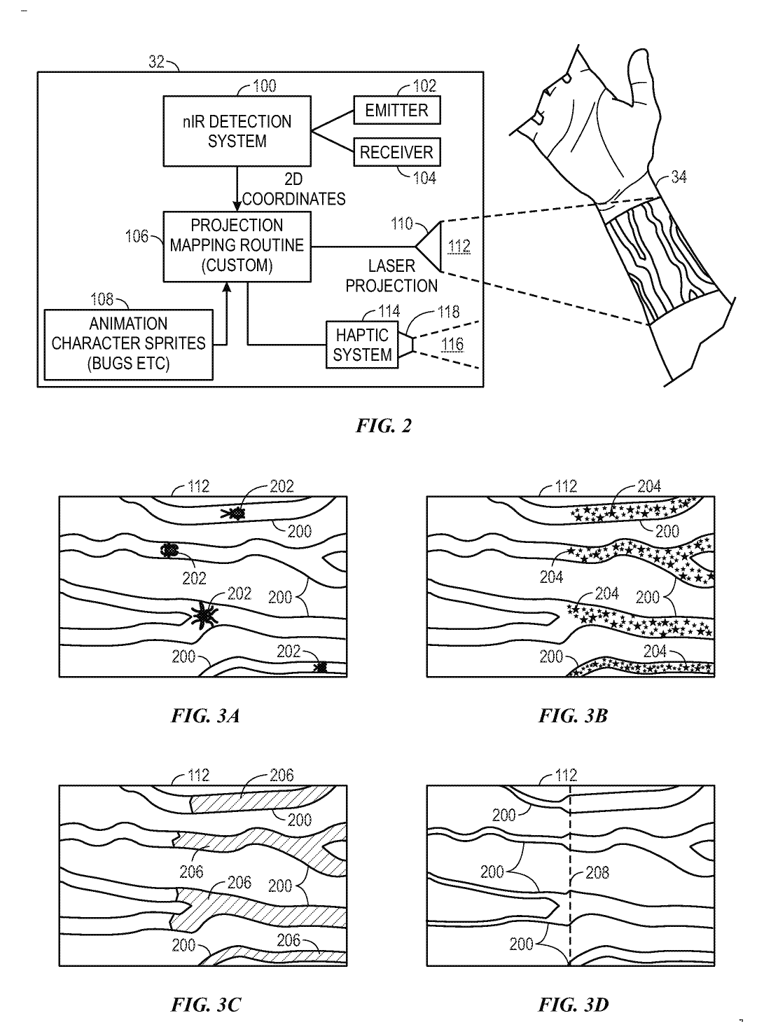

FIG. 2 is a schematic of the vascular effects system of FIG. 1. As shown, the vascular effects system 32 includes a near infrared (nIR) detection system 100, which includes an emitter 102 and a receiver 104 (e.g., an infrared camera). The emitter 102 emits infrared signals onto an object, such as a body part of the guest 34. For example, the emitter 102 may be a light-emitting diode (LED), an array of LEDs, or some other source of light in the infrared spectrum. The receiver 104 detects incoming infrared light from the emitter 102 (e.g., reflected from, or otherwise interacting with the guest’s body part) and generates a signal that can be processed to identify oxygen-rich and oxygen-depleted environments. Because blood flowing through veins is oxygen rich, the oxygen rich environments detected by the receiver 104 correspond to the veins (e.g., the vascular structure) of the guest’s body part. Accordingly, the signal received by the receiver 104 may be processed by the nIR detection system 100 to identify oxygen rich environments or regions within the guest’s vascular structure, identify the location of one or more veins within the guest’s vascular structure based on the oxygen rich environments, generate two-dimensional coordinates for the one or more veins of the vascular structure of the guest 34 underneath the vascular effects system 32.

A projection mapping routine 106, which may be represented by instructions stored in memory and executed by processing circuitry, receives the two-dimensional coordinates of the guest’s vascular structure and maps the guest’s vascular structure onto an area to be projected onto the guest’s body part. The projection mapping routine 106 retrieves one or more animation character sprites 108 stored in memory and adds the sprites (e.g., images and/or animations) to the projection. As previously described, the sprites may include, for example, bugs crawling on the guest’s veins, tattoos on the guest’s skin, magic, superpowers, or other supernatural forces/substances flowing through the guest’s veins, various colors of blood or other fluids flowing through the guest’s veins, poison flowing through the guest’s veins, veins getting bigger (e.g., swelling) or smaller, veins moving around, and so forth. The sprites may be defined by scripts or portions of code stored in memory. The scripts or portions of code may define various characteristics of the sprites, such as the shape of the sprite, the size of the sprite, how the sprite articulates as it moves, the speed of the sprite, and so forth. In some embodiments, one or more parameters of the sprites may be adjustable. Accordingly, the projection mapping routine 106 may add the animation character sprites 108 to the projection to simulate the sprites moving along the vascular structure (e.g., moving along the veins) in accordance with the script, portion of code, and/or one or more customizable parameters.

A laser projection system 110 projects images 112 onto the body part of the guest 34 to create the effect of the sprites 108 moving along the vascular structure of the guest 34 and to recreate (e.g., outline, highlight, mimic) the vascular structure itself. As the guest 34 moves their body part relative to the vascular effects system 32, the images projected onto the guest’s body part may update to reflect different portions of the guest’s vascular structure and different sprites and sprite activity.

As shown in FIG. 2, in some embodiments, the vascular effects system 32 may include a haptic system 114, which may be configured to release pressurized bursts of air 116 toward the skin of the guest via one or more nozzles 118. For example, the haptic system 114 may be configured to release pressurized bursts of air 116 at or near locations in which the projection system 110 projects images of sprites to make it seem to the guest like he or she can feel the sprites moving along his or her vascular structure. Accordingly, the haptic system 114 may include one or more adjustable nozzles 118 that can release pressurized bursts of air 116 in various directions and/or an array of nozzles 118 such that the haptic system 114 can deliver pressurized bursts of air 116 to specific locations.

In some embodiments, the vascular effects system 32 may be instantiated as a handheld device held by the guest 34 or a staff member. In some embodiments, the vascular effects system 32 may be fixed to a wall, an exhibit, or some other structure and the guest 34 may place his or her body part under the vascular effects system 32. Further, in some embodiments, the vascular effects system 32 may be contained within a single housing. In some embodiments, the various components of the vascular effects system 32 may not be contained in a single housing.

FIGS. 3A, 3B, 3C, and 3D illustrate examples of projections by the vascular effects system onto a guest’s body part. For Example, FIG. 3A illustrates an example in which the vascular effects system projects the vascular structure, including veins 200 of the guest, as sensed by the nIR detection system, as well as one or more bugs 202 crawling along the veins. The bugs may include, for example, spiders, flies, caterpillars, centipedes, moths, butterflies, mosquitos, scorpions, ladybugs, beetles, roaches, ants, bees, mantises, cicadas, silverfish, termites, fleas, ticks, dragonflies, lice, crickets, grasshoppers, hornets, yellowjackets, wasps, weevils, or other bugs, insects, small wildlife, and so forth. As previously described, the bugs 202 may be based on one or more sprites (e.g., still images or animations) saved in memory and defined by a script or a piece of code. The sprites, or the scripts/code that defines the sprites may be retrieved by the projection mapping routine 106 of the vascular effects system 32 shown in FIG. 2 and mapped over the guest’s vascular structure as sensed by the nIR detection system 100 of FIG. 2. Accordingly, the projection mapping routine may execute the code associated with the sprites to create the images 112 of the bugs crawling or otherwise moving along veins 200 of the vascular structure. However, it should be understood that bugs 202 depicted in FIG. 3A are merely an example and that the vascular effects system may be capable of creating other effects.

For example, FIG. 3B illustrates an example in which the vascular effects system projects the vascular structure, including veins 200, of the guest, as sensed by the nIR detection system, as well as magic 204 flowing through the veins 200. Though the magic 204 is shown via stars traveling along the veins 200, embodiments are also envisaged in which the magic is represented by something other than stars, such as sparkles, colored dust, mist/fog/vapor, different colored fluid, and so forth. As with the bugs 202 shown in FIG. 3A, the magic 204 may be based on sprites stored in memory whose parameters and/or characteristics may be defined by code.

Further, FIG. 3C illustrates an example in which the vascular effects system projects the vascular structure, including veins 200, of the guest, as sensed by the nIR detection system, as well as a fluid 206 flowing through the veins 200. Though the fluid shown in FIG. 3C is depicted with crosshatching, it should be understood that the fluid may be represented by fluid of a different color, texture, viscosity, etc.

FIG. 3D illustrates an example in which the vascular effects system projects the vascular structure, including veins 200, of the guest, as sensed by the nIR detection system, in which the veins are larger on one side of a line 208, and smaller on the other side of the line 208. The effect shown in FIG. 3D may be representative of enlarged veins 200, narrowed veins 200, and so forth. However, similar effects may also be used to simulate moving veins, pulsing veins, curling veins, sprouting veins, etc.

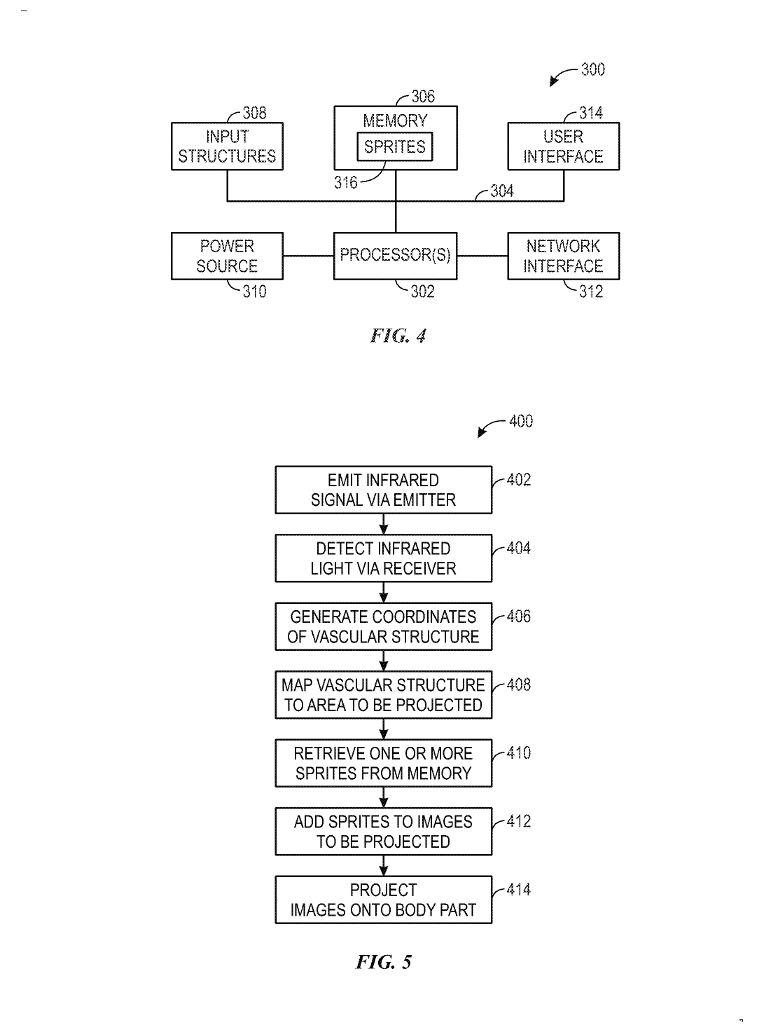

FIG. 4 illustrates a block diagram of example components of a computing device 300 that are configured to be used as the vascular effects system 32, the servers 42, or some other device within the amusement park 10 shown in FIG. 1. As used herein, a computing device 300 may be implemented as one or more computing systems including laptop, notebook, desktop, tablet, or workstation computers, as well as server type devices, network devices, such as routers, switches, edge devices, internet of things (IoT) devices, microprocessors, or portable, communication type devices, such as cellular telephones and/or other suitable computing devices.

As illustrated, the computing device 300 includes various hardware components, such as one or more processors 302, one or more busses 304, memory 306, input structures 308, a power source 310, a network interface 312, a user interface 314, and/or other computer components useful in performing the functions described herein.

The one or more processors 302 (e.g., processing circuitry) may include, in certain implementations, microprocessors configured to execute instructions stored in the memory 306 or other accessible locations. Alternatively, the one or more processors 302 may be implemented as application-specific integrated circuits (ASICs), field-programmable gate arrays (FPGAs), and/or other devices designed to perform functions discussed herein in a dedicated manner. As will be appreciated, multiple processors 302 or processing components may be used to perform functions discussed herein in a distributed or parallel manner.

The memory 306 may encompass any tangible, non-transitory medium for storing data or executable routines. Although shown for convenience as a single block in FIG. 4, the memory 306 may encompass various discrete media in the same or different physical locations. For example, the memory may store code for one or more sprites 316, and/or program code to be executed by the processors 302. The one or more processors 302 (e.g., processing circuitry) may access data in the memory 306 via one or more busses 304. In some embodiments, the various components may communicate with one another wirelessly.

The input structures 308 may allow a user to input data and/or commands to the device 300 and may include mice, touchpads, touchscreens, keyboards, controllers, and so forth. The power source 310 can be any suitable source for providing power to the various components of the computing device 300, including line and battery power. In the depicted example, the device 300 includes a network interface 312. Such a network interface 312 may allow communication with other devices on a network using one or more communication protocols. In the depicted example, the device 300 includes a user interface 314, such as a display that may display images or data provided by the one or more processors 302. The user interface 314 may include, for example, a monitor, a display, and so forth. As will be appreciated, in a real-world context a processor-based system, such as the computing device 300 of FIG. 4, may be employed to implement some or all of the present approach, such as performing the functions of the vascular effects system 32 and the servers 42, shown in FIGS. 1 and 2, as well as other memory-containing devices.

FIG. 5 is a flow chart of a process 400 for creating vascular effects. At 402, the process 400 emits (e.g., via an emitter or an array of emitters) infrared (IR) signals, such as light in the infrared spectrum. The infrared signals may be directed toward a body part of a guest, and/or a body part of a guest may be placed in the infrared signals being emitted. At 404, the process 400 detects incoming infrared light via a receiver. The incoming infrared light detected by the receiver may be reflected by the body part of the guest or otherwise interact with the body part of the guest. The receiver generates a signal based on the reflected infrared light that can be processed to identify oxygen rich environments and distinguish the oxygen rich environments from oxygen depleted environments. Because blood flowing through veins of a guest’s body part is oxygen rich, the oxygen-rich environments identified by the receiver correspond to the vascular structure of the body part of the guest.

At 406, the signal output by the receiver is processed to generate coordinates of the vascular structure of the guest’s body part. For example, the process 400 may generate a set of coordinates that represent an outline of the guest’s vascular structure, points where the guest’s vascular structure is, boundaries/edges of the guest’s vascular structure, and so forth. At 408, the process 400 maps the guest’s vascular structure to the area to be projected onto the guest’s body part based on the coordinates of the vascular structure generated at 406.

At 410, the process retrieves one or more sprites from memory. As previously described, the sprites may include images and/or animations defined by scripts or portions of code that define various characteristics of the sprites, such as the shape of the sprite, the size of the sprite, how the sprite articulates as it moves, the speed of the sprite, and so forth. In some embodiments, one or more parameters of the sprites may be adjustable.

At 412, the sprites are added to the images to be projected onto the body part of the guest. Accordingly, the process 400 may add the sprites to the projection to simulate the sprites moving along the guest’s vascular structure (e.g., moving along the veins) in accordance with the script, portion of code, and/or one or more customizable parameters. Accordingly, the sprites may be mapped onto the guest’s vascular structure such that the sprites are positioned along veins within the guest’s vascular structure such that the sprites appear to be along, within, or expanding the veins. The sprites may include, for example, bugs crawling on/in the guest’s veins, tattoos on the guest’s skin, magic, superpowers, or other supernatural forces/substances flowing through the guest’s veins, various colors of blood or other fluids flowing through the guest’s veins, poison flowing through the guest’s veins, veins getting bigger (e.g., swelling) or smaller, veins moving around, and so forth. For example, the sprites may include spiders, flies, caterpillars, centipedes, moths, butterflies, mosquitos, scorpions, ladybugs, beetles, ants, bees, mantises, cicadas, silverfish, termites, fleas, dragonflies, lice, crickets, grasshoppers, hornets, yellowjackets, wasps, weevils, or other bugs, insects, small wildlife, and so forth. In an embodiment, the sprites may include stars, sparkles, colored dust, mist/fog/vapor, different colored fluid, and so forth to represent magic flowing through a guest’s veins. Further, the sprites may simulate a fluid of a specific color, texture, or viscosity flowing through the veins, as well as veins swelling, narrowing, pulsing, etc. At 414, the images are projected onto the body part of the guest. As previously discussed, projecting images on the guest’s skin may be accompanied by releasing pressurized bursts of air toward the guest’s skin via a haptic system to simulate the feeling of the sprites moving along the vascular structure of the guest.

In some embodiments, the process 400 may operate in real time or near-real time such that the images projected by the process 400 change based on the body part, and correspondingly, the vascular structure of the body part moving underneath the vascular effects system. Accordingly, the process may return to 402 and emit an infrared signal from the emitter.

The present disclosure is directed to techniques for creating vascular effects in a curated environment (e.g., amusement parks, museums, historical sites, zoos, parks, art galleries, fairs, trade shows, conferences, conventions, expos, festivals, and so forth) using a vascular effects system. The vascular effects system includes an emitter, a receiver, a projector, and a processor-based computing device. The emitter emits infrared light onto a body part of a guest, such as an arm, a hand, or a leg. The receiver detects the infrared light from the body part of the guest and outputs a signal that may be processed to identify the vascular structure of the body part of a guest underneath the skin. The computing device receives the signal from the receiver, generates two-dimensional coordinates of the vascular structure, and maps the vascular structure to an area to be projected onto the guest’s body part. The computing device retrieves code defining one or more sprites from memory and adds the sprites to the projection. The sprites may include images and/or animations that move along (e.g., within, along the outside of, expanding or morphing) the vascular structure. For example, the sprites may represent bugs crawling on the guest’s veins, tattoos on the guest’s skin, magic, superpowers, or other supernatural forces/substances flowing through the guest’s veins, various colors of blood or other fluids flowing through the guest’s veins, poison flowing through the guest’s veins, veins getting bigger (e.g., swelling) or smaller, veins pulsing, veins moving around, and so forth. As the guest’s body part moves relative to the vascular effect system, the projected images may update to show a different portion of the guest’s vascular structure. By utilizing the disclosed techniques, guest experiences in curated spaces may be improved by increasing the number of interactive experiences. Accordingly, use of vascular vision effects may increase guest engagement and satisfaction.

While only certain features of the invention have been illustrated and described herein, many modifications and changes will occur to those skilled in the art. It is, therefore, to be understood that the appended claims are intended to cover all such modifications and changes as fall within the true spirit of the invention.

The techniques presented and claimed herein are referenced and applied to material objects and concrete examples of a practical nature that demonstrably improve present technical field and, as such, are not abstract, intangible or purely theoretical. Further, if any claims appended to the end of this specification contain one or more elements designated as “means for (perform)ing (a function)…” or “step for (perform)ing (a function)…”, it is intended that such elements are to be interpreted under 35 U.S.C. 112(f). However, for any claims containing elements designated in any other manner, it is intended that such elements are not to be interpreted under 35 U.S.C. 112(f).

CLAIMS:

1. A vascular effects system, comprising:
an emitter configured to emit infrared light toward a body part of a guest;
a receiver configured to:
receive the infrared light from the body part of the guest; and
generate a signal indicative of the received infrared light;
a projector configured to project images onto the body part of the guest;
processing circuitry; and
memory, accessible by the processing circuitry, the memory storing:
a portion of code representing a sprite; and
instructions that, when executed by the processing circuitry, are configured to cause the processing circuitry to perform operations comprising:
receiving, from the receiver, the signal indicative of the received infrared light;
generating, based on the signal indicative of the received infrared light, a set of coordinates corresponding to a vascular structure of the body part of the guest;
retrieving, from the memory, the portion of code representing the sprite;
generating, based on the portion of code, an image comprising the vascular structure of the body part and the sprite; and
projecting, via the projector, the image comprising the vascular structure and the sprite onto the body part of the guest.

2. The system of claim 1, wherein generating the set of coordinates corresponding to the vascular structure of the body part of the guest comprises:
identifying, based on the signal indicative of the received infrared light, one or more oxygen rich regions within the vascular structure; and
identifying a location of one or more veins within the vascular structure corresponding to the one or more oxygen rich regions within the vascular structure; and
generating the set of coordinates based on the location of one or more veins within the vascular structure.

3. The system of claim 1, wherein the emitter comprises a light emitting diode (LED).

4. The system of claim 1, wherein the sprite comprises a representation of one or more bugs.

5. The system of claim 4, wherein the one or more bugs comprise a spider, a fly, a caterpillar, a centipede, a moth, a butterfly, a mosquito, a scorpion, a ladybug, a beetle, an ant, a bee, a mantis, a cicada, a silverfish, a termite, a flea, a tick, a dragonfly, a louse, a cricket, a grasshopper, a hornet, a yellowjacket, a wasp, a weevil, or any combination thereof.

6. The system of claim 1, wherein the sprite comprises a depiction of magic flowing through the vascular structure of the body part of the guest.

7. The system of claim 1, wherein the sprite comprises a depiction of fluid flowing through the vascular structure of the body part of the guest.

8. The system of claim 1, wherein the sprite comprises a depiction of one or more veins within the vascular structure of the body part of the guest enlarging, narrowing, or both.

9. The system of claim 1, wherein the sprite comprises a depiction of one or more veins within the vascular structure of the body part of the guest moving.

10. The system of claim 1, wherein the sprite comprises a depiction as moving along the vascular structure.

11. The system of claim 1, wherein the operations comprise mapping the vascular structure to an area of the body part of the guest.

12. The system of claim 11, wherein mapping the vascular structure to the area of the body part of the guest comprises executing a projection mapping routine stored in the memory.

13. The system of claim 1, wherein the vascular effects system comprises a handheld device configured to be held over the body part of the guest.

14. The system of claim 1, wherein the vascular effects system is configured to be installed in a fixed position, wherein the body part of the guest is placed in the infrared light emitted by the emitter.

15. A method of providing vascular effects, the method comprising:
emitting infrared light toward a body part of a guest;
receiving the infrared light from the body part of the guest;
generating a signal indicative of the received infrared light;
generating, based on the signal indicative of the received infrared light, a set of coordinates corresponding to a vascular structure of the body part of the guest;
mapping the vascular structure to a body part of the guest;
retrieving a portion of code representing a sprite;
generating, based on the portion of code, a series of images comprising the vascular structure of the body part of the guest and the sprite; and
projecting the series of images comprising the vascular structure and the sprite onto the body part of the guest.

16. The method of claim 15, wherein generating the set of coordinates corresponding to the vascular structure of the body part of the guest comprises:
identifying, based on the signal indicative of the received infrared light, one or more oxygen rich regions within the vascular structure; and
identifying a location of one or more veins within the vascular structure corresponding to the one or more oxygen rich regions within the vascular structure; and
generating the set of coordinates based on the location of one or more veins within the vascular structure.

17. The method of claim 15, comprising releasing, via a haptic system, one or more pressurized bursts of air toward the body part of the guest corresponding to the series of images comprising the vascular structure and the sprite onto the body part of the guest.

18. A non-transitory computer readable medium storing instructions that, when executed by processing circuitry, cause the processing circuitry to perform operations comprising:
receiving a signal indicative of infrared light sensed from a body part of a guest;
generate, based on the signal indicative of the sensed infrared light, a set of coordinates corresponding to a vascular structure of the body part of the guest;
mapping the vascular structure to an area of the body part of the guest;
retrieving a portion of code representing a sprite;
generating, based on the portion of code, an image comprising the vascular structure of the area of the body part of the guest and the sprite; and
transmitting the image to a projector to project the image onto the body part of the guest.

19. The non-transitory computer readable medium of claim 18, wherein the operations comprise generating a series of images, including the image, comprising the vascular structure of the area of the body part of the guest and the sprite moving along the vascular structure of the area of the body part of the guest.

20. The non-transitory computer readable medium of claim 18, wherein generating the set of coordinates corresponding to the vascular structure of the body part of the guest comprises:
identifying, based on the signal indicative of the sensed infrared light, one or more oxygen rich regions within the vascular structure; and
identifying a location of one or more veins within the vascular structure corresponding to the one or more oxygen rich regions within the vascular structure; and
generating the set of coordinates based on the location of one or more veins within the vascular structure.
